(19) United States
(12) Patent Application Publication
(10) Pub. No.: US 2012/0129769 A1
(43) Pub. Date: May 24, 2012

(54) ORALLY ADMINISTERABLE PHARMACEUTICAL PREPARATION CONTAINING INSULIN

(76) Inventors: Zoltán Szilvássy, Debrecen (HU); Barna Peitl, Györ (HU); József Németh, Pécs (HU)

(21) Appl. No.: 13/387,212

(22) PCT Filed: Aug. 2, 2010
(86) PCT No.: PCT/IB10/53499

§ 371 Date: Feb. 2, 2012

(30) Foreign Application Priority Data
Aug. 3, 2009 (HU) P0900482

(51) Int. Cl. A61K 38/28; A61P 3/10

(52) U.S. Cl. 514/6.5

(57) ABSTRACT

The subject of the invention is an orally administerable pharmaceutical preparation containing a combination of biotechnologically produced human recombinant insulin and/or modified insulin or an analogue and/or derivative thereof, a protease inhibitor and a high molecular weight (natural) protein. The invention relates to a method for the production of the pharmaceutical preparation as well. The subject of the invention also covers the use of the pharmaceutical preparation and a method for the treatment of diabetes in mammals.

The subject of the invention is an orally administerable pharmaceutical preparation containing a combination of biotechnologically produced human recombinant insulin and/or modified insulin or an analogue and/or derivative thereof, a protease inhibitor and a high molecular weight (natural) protein. The invention relates to a method for the production of the pharmaceutical preparation as well. The subject of the invention also covers the use of the pharmaceutical preparation and a method for the treatment of diabetes in mammals.

In the development of orally administerable insulin preparations two basic problems need to be solved: the inhibition of the degradation of insulin of peptide nature, and getting it through the intestinal barrier.

According to the literature there have been numerous attempts to develop orally administerable pharmaceutical preparations containing insulin. Essentially, these preparations differ from one another in that they contain different substances in addition to insulin in order to inhibit the enzymatic inactivation, to promote the absorption and resorption of insulin.

Document No. EP1454631 describes a pharmaceutical preparation containing a therapeutically effective quantity of insulin and crystalline dextran microparticles in aqueous suspension. The preparation provides a controlled insulin release that can be single-phase or multi-phase.

Document No. US1993005970 discloses a pharmaceutical preparation containing insulin covalently bound to an oligomer.

The pharmaceutical preparation disclosed in international publication document No. WO0033866 contains insulin in a non-aqueous hydrophilic medium, mixed with long-chain PEG species, in the form of a suspension.

International publication document No. WO9636352 describes an insulin preparation suitable for oral or nasal administration, containing at least two compounds promoting absorption, e.g. Na-salicylate, Na-lauryl sulphate, oleic acid, linoleic acid, lecitin, etc.

The subject of U.S. Pat. No. 5,438,040 is an orally administerable pharmaceutical preparation containing a conjugated insulin complex, where the insulin is covalently bound to a physiologically compatible polyalkylene glycol derivative, which is stable and water-soluble, and at the same time does not degrade in the digestive system.

The preparation disclosed in Japanese patent application No. JP54028807 contains mucin as an additive, and an insulinase inhibitor.

The pharmaceutical preparation described in international publication document No. WO0166085 contains insulin, alkali metal (C8 to C22 alkyl) sulphate, water or ethanol as a solvent, a phenolic compound, an antioxidant and a protease inhibitor—e.g. bacitracin or a derivative thereof, soy trypsin or aprotinin.

International publication document No. WO9310767 provides a solution for the problem of the oral administration of any peptide-type active ingredient that is enzymatically inactivated in the gastrointestinal tact. In the case of insulin this aim is achieved by incorporating the insulin into a gelatin matrix. The gelatin allows the active ingredient to be absorbed in the small and large intestines in such a way that it is not exposed to the degrading effect of peptidase for a long time.

The pharmaceutical preparation disclosed in document No. EP0127535 contains insulin, bile acid and a protease inhibitor. The bile acid promotes absorption, the protease inhibitor protects the insulin from proteolysis. The orally administered preparation quickly passes through the stomach, and it is released and quickly absorbed in the intestines.

European patent application No. EP0351651 discloses a preparation suitable for oral and buccal administration containing, in addition to insulin, polyoxyethylene glycol-carboxyl acid-glyceride ester as an absorption-promoting substance, and a carrier substance.

The preparation disclosed in U.S. Pat. No. 3,172,814 contains insulin and anhydro-formaldehyde aniline in order to prevent a decrease in the effect of insulin.

The preparation according to international publication document No. WO2007121318 contains insulin and sodium 4-CNAB as a carrier substance, which are lyophilized together, then the obtained powder is tabletted or filled into gelatin capsules.

According to international publication document No. WO9843615 a swellable hydrogel matrix is used, which is the copolymer of methacrylic acid and polyalkylene glycol, and allows the insulin to be released only when it reaches the small intestine. The polymer also inhibits the activity of proteolytic enzymes in the intestines, and helps insulin to remain active for a long time before absorption.

The preparations known so far are generally characterized by the fact that the bioavailability of insulin is low, only a small amount is absorbed from the gastrointestinal tract, it quickly degrades and fails to affect the blood sugar level.

The aim of the invention was to develop an orally administerable pharmaceutical preparation containing insulin, with better bioavailability than those known so far.

The set aim was achieved with a combination insulin, a protease-inhibiting substance and a high molecular weight natural protein. It is important that both the protease inhibiting substance and the protein shall have intestinal carriers, so that both can pass through the intestinal wall and with the appropriate carrier molecules can get through the insulin of peptide nature as well.

The invention relates to an orally administerable pharmaceutical preparation containing a combination of biotechnologically produced human recombinant insulin and/or modified insulin or an analogue and/or derivative thereof, a protease inhibitor and a high molecular weight (natural) protein.

The human insulin is an analogue with Asp, Lys, Leu, Val or Ala at position B28 and Lys or Pro at position B29; or des(B28-B30), des(B27) or des(B30) human insulin.

According to a preferred embodiment of the invention the protease inhibitor is ε-amino-caproic acid and the high molecular weight natural protein is casein.

The pharmaceutical preparation according to the invention contains 40-100 IU of human recombinant insulin, 100-1000 mg of ε-amino-caproic acid and 1-100 mg of casein, and pharmaceutically acceptable carrier and additive substances.

The pharmaceutical preparation according to the invention can be used for the treatment of (type 1 and 2) diabetes in mammals.

The pharmaceutical preparation according to the invention can also be used advantageously for the treatment of diabetes in pregnancy.

The invention also relates to the use of a combination of a therapeutically effective quantity of biotechnologically produced human recombinant insulin and/or modified insulin or an analogue and/or derivative thereof, ε-amino-caproic acid and casein for the production of an orally administerable pharmaceutical preparation suitable for the treatment of (type 1 and 2) diabetes in mammals.

The subject of the invention also covers a method for the production of the orally administerable pharmaceutical preparation, according to which 40-100 IU of biotechnologically produced human recombinant insulin and/or modified insulin or an analogue and/or derivative thereof, 100-1000 mg of ε-amino-caproic acid and 1-100 mg of casein, mixed with pharmaceutically acceptable carrier and additive substances, are formulated into an orally administerable dosage form.

The orally administerable pharmaceutical preparation can be a capsule, a tablet or a film-coated tablet.

The invention also relates to a method for the treatment of (type 1 and 2) diabetes in mammals, according to which patients are given an orally administerable pharmaceutical preparation containing a therapeutically effective quantity of biotechnologically produced human recombinant insulin and/or modified insulin or an analogue and/or derivative thereof, ε-amino-caproic acid and casein. More precisely, patients are given a pharmaceutical preparation containing 40-100 IU of human recombinant insulin, 100-1000 mg of ε-amino-caproic acid and 1-100 mg of casein.

The pharmaceutical preparation according to the invention is characterized by a bioavailability of over 30%.

Our invention is described in more detail by means of FIGS. 1-3 and the results of the tests presented in the examples.

FIG. 1: Insulin (100 μM; native) stability in the presence of an equivalent quantity of insulin/ε-amino-caproic acid (acepramin, Sigma, MO) in a solution containing α-chymotrypsin (1.5 μg/10 μl).

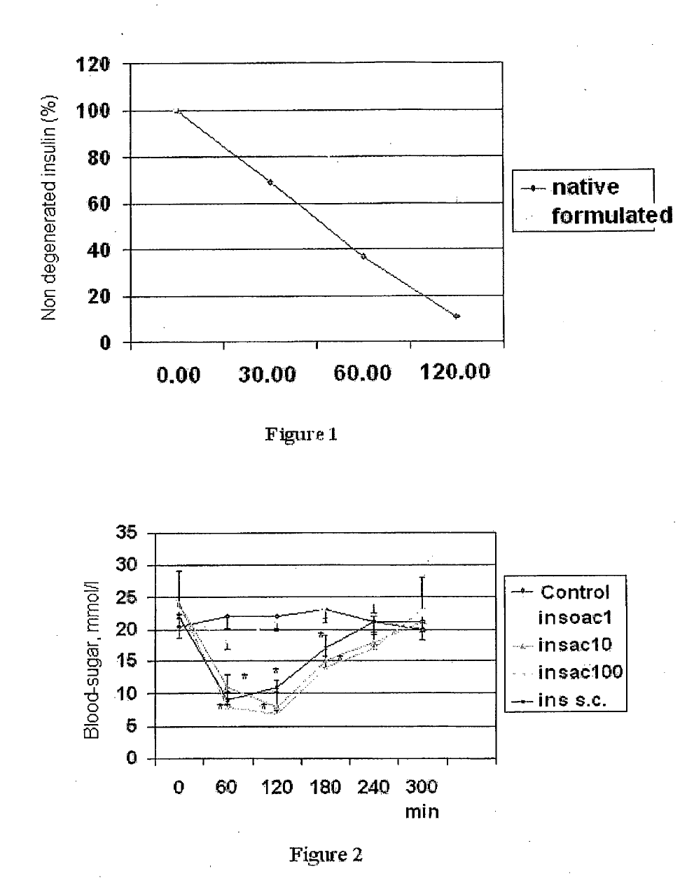

FIG. 2: The effect of a single oral dose of insulin (10 IU/kg) on the blood sugar level in streptozotocin (50 mg/kg i.v.) induced diabetes. In comparison to subcutaneous insulin (10 IU/kg). * a significant difference compared to the control. (p<0.05). n=8 by group. Abbreviations: insac1 (oral insulin with 1 mg/kg of ε-amino-caproic acid; insac10: oral insulin with 10 mg/kg of ε-amino-caproic acid; insac100: oral insulin with 100 mg/kg of ε-amino-caproic acid; ins s.c.: subcutaneous insulin.

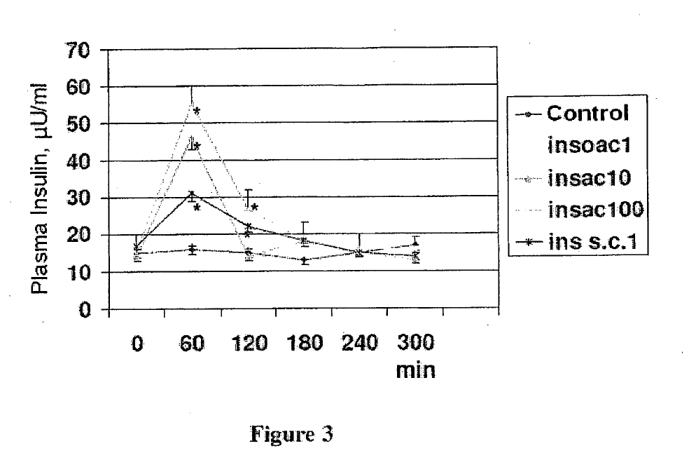

FIG. 3: The effect of a single oral dose of insulin (10 IU/kg) on plasma insulin immunoreactivity in streptozotocin (50 mg/kg i.v.) induced diabetes. In comparison to subcutaneous insulin (10 IU/kg). * significant difference compared to the control. (p<0.05). n=8 by group.

The data in FIG. 1 show that after 120 minutes 60% of the formulated insulin is intact, while more than 80% of the native insulin has degraded.

The data in FIGS. 2 and 3 prove that the formulated oral insulin increases the plasma insulin level, and effectively reduces the blood sugar level in insulin deficiency diabetes. On the basis of the experiments, with the same standard insulin (10 IU/kg) an ε-amino-caproic acid content of 100 mg/kg does not provide an advantage compared to 10 mg/kg. The “60-minute” values for subcutaneous insulin are probably already declining plasma concentration values.

### EXAMPLES

### Example 1

### The Possibility of Using Oral Insulin by Means of ε-amino-caproic Acid Carrier Molecules

Male Wistar rats (Charles-River Laboratories, Budapest, Hungary) were used for the experiments. Before the experiment the animals were starved for 16 hours. The experiments started between 8 and 9 h in the morning. 2×6 groups were formed in a random manner, with 4 animals by group. The animals were pretreated through a feeding probe according to the followings: group 1: with 1 g/kg of ε-amino-caproic acid; group 2: with 0.1 U/kg of insulin; group 3: with 0.1 U/kg of insulin and 1 g/kg of ε-amino-caproic acid; group 4: with 1.0 U/kg of insulin; group 5: with 1.0 U/kg of insulin and 1 g/kg of ε-amino-caproic acid; and group 6: with a solvent. The blood sugar and plasma insulin levels were determined from arterial blood drawn after 15 minutes following the treatment for the first 6 groups and after 60 minutes for the second 6 groups. The obtained results are summarized in Table 1:

TABLE 1

| | |
|---|---|
| Blood sugar | Plasma |
| (mmol/l) | insulin (μU/ml) |

15-minute values

| | | |
|---|---|---|
| Solvent-treated | 5.52 ± 0.23 | 7.8 ± 2.31 |
| 1 g/kg of ε-amino-caproic acid | 5.43 ± 0.55 | 7.3 ± 2.52 |
| 0.1 IU/kg of insulin | 5.96 ± 0.21 | 10.75 ± 2.21 |
| 0.1 IU/kg of insulin + 1 g/kg of ε-amino- | 6.45 ± 0.53 | 15.85 ± 4.72 |
| caproic acid | | |
| 1.0 IU/kg of insulin | 5.77 ± 0.22 | 38.25 ± 6.95 |
| 1.0 IU/kg of insulin + 1 g/kg of ε-amino- | 6.42 ± 0.36 | 24.57 ± 4.99 |
| caproic acid | | |

60-minute values

| | | |
|---|---|---|
| Solvent-treated | 5.41 ± 0.45 | 8.21 ± 1.98 |
| 1 g/kg of ε-amino-caproic acid | 5.37 ± 0.15 | 7.76 ± 2.23 |
| 0.1 IU/kg of insulin | 4.97 ± 0.40 | 10.8 ± 1.68 |
| 0.1 IU/kg of insulin + 1 g/kg of ε-amino- | 5.6 ± 0.08 | 10.3 ± 2.53 |
| caproic acid | | |
| 1.0 IU/kg of insulin | 4.67 ± 0.41 | 23.87 ± 4.05 |
| 1.0 IU/kg of insulin + 1 g/kg of ε-amino- | 5.72 ± 0.43 | 28.12 ± 3.84 |
| caproic acid | | |

### Example 2

### The Effect of Acepramin on the Absorption of Insulin with Standard Casein

Healthy male Wistar rats (230-250 g) were given an ε-amino-caproic acid—human recombinant insulin mixture with standard casein intraduodenally. The endpoint of the measurement was the blood sugar measured with the glucose oxidase method, and the plasma insulin immunoreactivity measured with radioimmunoassay 15 and 60 minutes after the administration of the insulin-acepramin formulation (aqueous suspension).

The results of the experiments are shown in Table 2:

Data: mean±S.D. with n=8 by group. Statistics: t-test with Bonferroni correction, after ANOVA.

TABLE 2

| | Blood | Plasma |
|---|---|---|
| | sugar (mmol/l) | insulin (μU/ml) |
| 15^{th} minute | | |
| Solvent | 7.3 ± 0.34 | 15.9 ± 3.35 |
| 100 mg/kg of ε-amino-caproic acid | 6.9 ± 1.41 | 17.1 ± 3.62 |
| 0.1 IU/kg of insulin | 7.9 ± 0.92 | 14.4 ± 1.51 |
| 0.1 IU/kg of insulin + 100 mg/kg of | 5.55 ± 0.71* | 19.8 ± 1.7* |
| Acepramin | | |
| 1.0 IU/kg of insulin | 7.7 ± 2.31 | 27.5 ± 3.95* |
| 1.0 IU/kg of insulin + 100 mg/kg of ε- | 4.9 ± 0.86* | 44.1 ± 4.52* |
| amino-caproic acid | | |
| 60^{th} minute | | |
| Solvent | 7.4 ± 0.52 | 14.8 ± 2.53 |
| 100 mg/kg of ε-amino-caproic acid | 7.3 ± 0.95 | 16.7 ± 3.00 |
| 0.1 IU/kg of insulin | 7.9 ± 0.83 | 17.3 ± 2.94 |
| 0.1 IU/kg of insulin + 100 mg/kg of ε- | 6.1 ± 0.59* | 21.4 ± 2.62* |
| amino-caproic acid | | |
| 1.0 IU/kg of insulin | 6.9 ± 0.62 | 17.1 ± 2.06 |
| 1.0 IU/kg of insulin + 100 mg/kg of ε- | 4.9 ± 0.43* | 31.2 ± 2.79* |
| amino-caproic acid | | |

*significant change, p < 0.05
The asterisk and the highlighted data show that the oral insulin was absorbed and reduced the blood sugar level.

### Example 3

### “In Vitro” Stability

In vitro stability means the biodegradation of a primitive formulation of the insulin-acepramin mixture in the presence of a protein degrading enzyme, as compared to native insulin (results of reverse-phase HPLC tests).

### Example 4

### Bioavailability and Effectiveness in Experimental Diabetes

Experimental diabetes was induced in Sprague-Dawley male rats (230-250 g) with a single intravenous dose of streptozotocin. After 10 days the experiments were continued with the animals having a fasting (after 12 hours of starving) blood sugar level higher than 15 mmol/l. The animals were given oral or parenteral (s.c.) insulin (10 IU/kg), then the blood sugar level (data in Table 2) and the plasma insulin immunoreactivity (data in Table 3) were measured.

The pharmaceutical preparation according to the invention is nearly equivalent to the subcutaneously administered insulin in terms of blood sugar reducing effect, it is suitable for reducing abnormally high blood sugar levels, for treating diabetic mammals.

The pharmaceutical preparation has an insulin sensitization effect to subcutaneously administered insulin.

The elimination half life of the pharmaceutical preparation is about 40 minutes in rats.

The pharmaceutical preparation exhibits no subchronic toxicity.

1. An orally administerable pharmaceutical preparation, wherein it contains a combination of biotechnologically produced human recombinant insulin and/or modified insulin or an analogue and/or derivative thereof, a protease inhibitor and a high molecular weight natural protein.

2. The pharmaceutical preparation according to claim 1, wherein the human insulin is an analogue with Asp, Lys, Leu, Val or Ala at position B28 and Lys or Pro at position B29; or des(B28-B30), des(B27) or des(B30) human insulin.

3. The pharmaceutical preparation according to claim 1, wherein the protease inhibitor is ε-amino-caproic acid.

4. The pharmaceutical preparation according to claim 1, wherein the high molecular weight natural protein is casein.

5. The pharmaceutical preparation according to claim 1, wherein it contains 40-100 IU of human recombinant insulin, 100-1000 mg of ε-amino-caproic acid and 1-100 mg of casein.

6. (canceled)

7. A method for the production of the orally administerable pharmaceutical preparation of claim 1, wherein 40-100 IU of biotechnologically produced human recombinant insulin and/or modified insulin or an analogue and/or derivative thereof, 100-1000 mg of ε-amino-caproic acid and 1-100 mg of casein, mixed with pharmaceutically acceptable carrier and additive substances, are formulated into an orally administerable dosage form.

8. The use of the pharmaceutical preparation according to claim 1 for the treatment of (type 1 and 2) diabetes in mammals.

9. The use of the pharmaceutical preparation according to claim 1 for the treatment of diabetes in pregnancy.

10. A method for the treatment of (type 1 and 2) diabetes in mammals, wherein patients are given an orally administerable pharmaceutical preparation containing a therapeutically effective quantity of biotechnologically produced human recombinant insulin and/or modified insulin or an analogue and/or derivative thereof, ε-amino-caproic acid and casein.

11. A method for the treatment of type 1 diabetes in mammals, wherein patients are given a pharmaceutical preparation containing 40-100 IU of human recombinant insulin, 100-1000 mg of ε-amino-caproic acid and 1-100 mg of casein.

12. The pharmaceutical preparation according to claim 1, wherein its bioavailability is over 30%.

13. The pharmaceutical preparation according to claim 2, wherein the high molecular weight natural protein is casein.

14. The pharmaceutical preparation according to claim 2, wherein it contains 40-100 IU of human recombinant insulin, 100-1000 mg of ε-amino-caproic acid and 1-100 mg of casein.

15. The pharmaceutical preparation according to claim 2, wherein its bioavailability is over 30%.
